(19) United States
(12) Patent Application Publication
(10) Pub. No.: US 2005/0074453 A1
(43) Pub. Date: Apr. 7, 2005

(54) Methods of preventing adhesions following laminectomies and other surgical procedures

(76) Inventor: Bret A. Ferree, Cincinnati, OH (US)

Correspondence Address: John G. Posa;Gifford, Krass, Groh, Sprinkle,, Anderson & Citkowski, P.C., 280 N. Old Woodward Ave, Suite 400, Birmingham, MI, 48009-5394, US

(21) Appl. No.: 10/678,011

(22) Filed: Oct. 2, 2003

(51) Int. Cl. A61K 39/395; C12N 15/86

(52) U.S. Cl. 424/145.1; 424/146.1; 435/456

(57) ABSTRACT

Adhesions and scar formation following a surgical procedure are controlled by providing a human recombinant phage antibody, and introducing the antibody onto or into an area of the body following the procedure to inhibit adhesions, or scar formation. According to one embodiment, the antibody is used to prevent the formation of scar tissue following spinal surgery. This may be carried out by placing the antibody over the dura lining the spinal nerves and spinal cord. Alternatively, the antibody may be used to inhibit adhesions following abdominal surgery, or placed around the great vessels following an anterior approach to the spine or other regions. Importantly, the antibody may be used to inhibit adhesion formation adjacent to areas where growth factors are used to stimulate healing. The invention may further include the step of protecting the growth factors and/or the area of the body where stimulated healing is desired from the antibodies to the growth factors. As a further option, other medications or therapeutic substances may be added to the antibody(ies) to enhance healing or effectiveness.

### FIELD OF THE INVENTION

This invention relates generally to therapeutic treatments and, in particular, to methods of preventing adhesions following surgical procedures.

### BACKGROUND OF THE INVENTION

Over a half million patients undergo lumbar laminectomies each year. Surgeons perform laminectomies to treat herniated lumbar discs, tumors, and spinal stenosis. At least five percent of the patients who undergo laminectomy will require additional spinal operations. Scar tissue following the first spinal operation makes repeat spinal operations are more difficult. The complication rate for repeat lumbar laminectomy is higher. Most of the increased risks of repeat operations can be attributed to scar tissue. The scar tissue increases the likelihood of the surgeon damaging the nerves and the likelihood of the patient developing a postoperative spinal fluid leak. Accordingly, any technique capable of inhibiting scar formation following such procedures would appeal to patients and surgeons.

### SUMMARY OF THE INVENTION

This invention resides in a method of controlling adhesions following a surgical procedure by providing a human recombinant phage antibody, and introducing the antibody onto or into an area of the body following the procedure to inhibit adhesions, or scar formation. Various substances and formulations may be used, including antibodies to one or more of the following:
Transforming Growth Factors-Beta (TGF-Beta),
Platelet Derived Growth Factors (PDGF),
Insulin-like Growth Factors (IGF),
Transforming Growth Factor-Alpha (TGF-alpha),
Epidermal Growth Factor (EGF),
Interleukins,
Leukocyte Derived Growth Factor (LDGF),
Fibroblastic Growth Factors (FGF),
Vascular Endothelial Growth Factor (VEGF),
Heparin-Binding Epidermal Growth Factor (HB-EGF),
Bone Morphogenetic Proteins (BMP), and
other cytokines associated with wound healing.

According to one embodiment, the antibody is used to prevent the formation of scar tissue following spinal surgery. This may be carried out by placing the antibody over the dura lining the spinal nerves and spinal cord. Alternatively, the antibody may be used to inhibit adhesions following abdominal surgery, or placed around the great vessels following an anterior approach to the spine or other regions.

Importantly, the antibody may be used to inhibit adhesion formation adjacent to areas where growth factors are used to stimulate healing. In such instances, the method may include adding growth factors to an area of the body where bone or tissue regeneration is desired, and using antibodies to the growth factors with respect to areas where adhesion prevention is desired. For instance, the growth factors may be introduced in conjunction with spinal fusion and bone ingrowth for artificial disc replacement, with the antibodies to the growth factors being targeted to the dura, nerves, and spinal cord to prevent adhesion.

The invention may further include the step of protecting the growth factors and/or the area of the body where stimulated healing is desired from the antibodies to the growth factors. This may include, for example, providing the growth factors in a slowly resorbing gel or polymer, and placing the slowly resorbing gel or polymer over the area where healing is desired. The growth factors may also be released slowly into the treatment area, by incorporating the growth factors into a hydrogel or other material or device to effectuate slow release. As a further option, other medications or therapeutic substances may be added to the antibody(ies) to enhance healing or effectiveness.

### DETAILED DESCRIPTION OF THE INVENTION

This invention broadly takes advantage of the discovery that antibodies to growth factors and other substances inhibit adhesion (scar formation). Such antibodies include, without limitation, antibodies to Transforming Growth Factors-Beta (TGF-Beta), Platelet Derived Growth Factors (PDGF), Insulin-like Growth Factors (IGF), Transforming Growth Factor-Alpha (TGF-alpha), Epidermal Growth Factor (EGF), Interleukins, Leukocyte Derived Growth Factor (LDGF), Fibroblastic Growth Factors (FGF), Vascular Endothelial Growth Factor (VEGF), Heparin-Binding Epidermal Growth Factor (HB-EGF), Bone Morphogenetic Proteins (BMP), and other cytokines important in wound healing.

In certain embodiments, human recombinant phage antibodies to TGF-B1 and TGF-B2, TGF-B3, Mannose-6-phosphate, and transglutaminase inhibitors are particularly advantageous. The invention may be used in any area of the body and following any type of surgical procedure. The preferred embodiments anticipate the use of these and similar materials to prevent the formation of scar tissue following spinal surgery. Such material would preferably be placed over the dura lining the spinal nerves and spinal cord.

According to this invention, the appropriate antibodies may also be used to inhibit adhesions, or scar formation, in other areas of the body. For example, they may be used to inhibit adhesions following abdominal surgery, placed around the great vessels after an anterior approach to the spine, or used to inhibit adhesion formation adjacent to areas where growth factors are used to stimulate healing. As such, growth factors may be added to areas of the spine where bone growth is desired (spinal fusion and bone ingrowth Artificial Disc Replacement (ADRs)) while using antibodies to the growth factors over areas where adhesion prevention is desired (dura, nerves, spinal cord). Similarly, growth factors could be used to accelerate an intestinal anastomosis while antibodies to the growth factors are used to inhibit intra-abdominal adhesions.

The growth factors, and the area of the body where stimulated healing is desired, may be protected from the antibodies to the growth factors. For example, the growth factors could be incorporated into a slowly resorbing gel or polymer that is placed over the area where healing is desired. The growth factors could be slowly released into the area as the material is resorbed. Hydrogels may be used for this purpose. Concentrated growth factors may also be released into the desired area after the antibodies to the growth factors are no longer present or active.

The antibodies and other substances may also be placed into a material that slowly releases the antibodies. Composite slow-release devices may be advantageous, such as the central portion of a hydrogel device containing growth factors. The outer portion of the hydrogel device could contain antibodies to growth factors, allowing the device to first release antibodies to growth factors, then release growth factors. Other medications or therapeutic substances may be added to the hydrogel or a layer of the hydrogel, including antibiotics or other medications. Materials other than hydrogels may also be used.

Antibodies to proteases that activate the latent form of TGF-beta could also be used to prevent adhesions. Similarly, other materials could be used to render the proteases inactive. For example, enzymes that deactivate the proteases could be used. The ph of the local environment could also be changed to deactivate the proteases.

The new embodiment is like a composite or three-piece device (like a sandwich). The center material acts as an impermeable barrier. For example, the center component (meat part of a sandwich) could be made of silatstic. A growth factor eluting component is used on one side of the barrier and an anti-growth factor eluting component is used on the other side of the barrier. Hydrogels could be used to slowly release the antibodies and the growth factors. The side of the device that elutes growth factors would be placed against the tissues were healing is desired (for example the anastomosis of intestines). The opposite side of the device inhibits adhesion formation. The sheet-like device could be placed over tissues like the dura and around tissues like the intestine. The antibodies to growth factors could also be released from cardiac stents and other implants.

Other therapeutic agents could be eluted, particularly from non-cardiac embodiments of the invention, include drugs that prevent the adhesion of platelets (including, but not limited to aspirin, Dipyridamole, Heparin, Coumadin, Protamine, and Hirudin), interupt cell reproduction (including, but not limited to Methotrexate, Colchicine, Azathioprine, Vincristine, VinBlastine, Fluorouracil, Adriamycin, and Mutamycin), prohibit inflammation (including, but not limited to glucocorticoids including dexamethasone), or block the receptors for the growth factors.

### REFERENCES

Logan A, Berry M, Gonzalez A M, Frautschy S A, Sporn M B, Baird A (1994) Effects of transforming growth factor beta 1 on scar production in the injured central nervous system of the rat. Eur J Neurosci 1: 355-363
Lucas P A, Warejcka D J, Young H E, Lee B Y (1996) Formation of abdominal adhesions is inhibited by antibodies to transforming growth factor-beta 1. J Surg Res 65: 135-138
Shah M, Foreman D M, Ferguson M W J (1994) Neutralising antibody to TGF-B1,2 reduces cutaneous scarring in adult rodents. J Cell Sci 107: 1137-1157
Shah M, Foreman D M, Ferguson M W J (1995) Neutralisation of TGF-B1 and TGF-B2 or exogenous addition of TGF-B3 to cutaneous rat wounds reduces scarring. J Cell Sci 108: 985-1002
Cordeiro M F, Gay J A, Khaw P T (1999) Human anti-transforming growth factor-beta2 antibody: a new glaucoma anti-scarring agent. Invest Ophthalmol Vis Sci 40: 225-2234

1. A method of controlling adhesions following a surgical procedure, comprising the steps of:
providing a human recombinant phage antibody; and
introducing the antibody onto or into an area of the body following the procedure to inhibit adhesions, or scar formation.

2. The method of claim 1, including antibodies to one or more of the following:
Transforming Growth Factors-Beta (TGF-Beta),
Platelet Derived Growth Factors (PDGF),
Insulin-like Growth Factors (IGF),
Transforming Growth Factor-Alpha (TGF-alpha),
Epidermal Growth Factor (EGF),
Interleukins,
Leukocyte Derived Growth Factor (LDGF),
Fibroblastic Growth Factors (FGF),
Vascular Endothelial Growth Factor (VEGF),
Heparin-Binding Epidermal Growth Factor (HB-EGF),
Bone Morphogenetic Proteins (BMP), and
other cytokines associated with wound healing.

3. The method of claim 2, including antibodies to TGF-β1, TGF-β2, TGF-β3, Mannose-6-phosphate, and transglutaminase inhibitors.

4. The method of claim 1, wherein the antibody is used to prevent the formation of scar tissue following spinal surgery.

5. The method of claim 1, wherein the antibody is placed over the dura lining the spinal nerves and spinal cord.

6. The method of claim 1, wherein the antibody is used to inhibit adhesions following abdominal surgery.

7. The method of claim 1, wherein the antibody is placed around the great vessels after an anterior approach to the spine.

8. The method of claim 1, wherein the antibody is used to inhibit adhesion formation adjacent to areas where growth factors are used to stimulate healing.

9. The method of claim 1, including the steps of:
adding growth factors to an area of the body where bone or tissue regeneration is desired; and
using antibodies to the growth factors with respect to areas where adhesion prevention is desired.

10. The method of claim 9, including the steps of:
adding growth factors in conjunction with spinal fusion and bone ingrowth for artificial disc replacement; and
using antibodies to the growth factors with respect to the dura, nerves, and spinal cord to prevent adhesion.

11. The method of claim 9, including the steps of:
adding growth factors to accelerate an intestinal anastomosis; and
using antibodies to inhibit intra-abdominal adhesions.

12. The method of claim 9, further including the step of:
protecting the growth factors and/or the area of the body where stimulated healing is desired from the antibodies to the growth factors.

13. The method of claim 9, further including the step of:
providing the growth factors in a slowly resorbing gel or polymer; and
placing the slowly resorbing gel or polymer over the area where healing is desired.

14. The method of claim 9, further including the step of:
slowly releasing the growth factors into the treatment area.

15. The method of claim 9, further including the step of:
incorporating the growth factors into a hydrogel to effectuate slow release.

16. The method of claim 9, further including the step of:
slowly releasing concentrated growth factors into the treatment area after the antibodies to the growth factors are no longer present or active.

17. The method of claim 9, further including the step of:
using a composite slow-release device to introduce a growth factor.

18. The method of claim 17, wherein the composite slow-release device is the central portion of a hydrogel device containing growth factors.

19. The method of claim 18, wherein the outer portion of the hydrogel device contains antibodies to growth factors, allowing the device to first release antibodies to growth factors, then release growth factors.

20. The method of claim 1, further including the step of adding other medications or therapeutic substances to the antibody.

21. The method of claim 1, including antibodies to proteases that activate the latent form of TGF-beta.

22. The method of claim 1, including the use of a protease inhibitor.

23. The method of claim 22, wherein the protease inhibitor is an enzyme that deactivates the protease.

24. The method of claim 22, wherein the ph of the local environment is altered to deactivate the protease.

25. The method of claim 1, wherein the antibodies are released from a cardiac stent or other implant.

26. The method of claim 20, wherein the other medications or therapeutic substances include drugs that prevent the adhesion of platelets, interrupt cell reproduction, prohibit inflammation, or block the receptors for the growth factors.
